(19) United States
(12) Patent Application Publication
(10) Pub. No.: US 2025/0000809 A1
(43) Pub. Date: Jan. 2, 2025

(54) Encapsulated biomolecules for intracellular delivery

(71) Applicant: Åbo Akademi, Åbo (FI)

(72) Inventors: Hongbo Zhang, Åbo (FI); Chang Liu, Åbo (FI); Xiaoyu Xu, Åbo (FI)

(21) Appl. No.: 18/575,330

(22) PCT Filed: Aug. 16, 2022
(86) PCT No.: PCT/FI2022/050532

§ 371 Date: Dec. 29, 2023

(30) Foreign Application Priority Data
Aug. 16, 2021 (FI) 20215856

(51) Int. Cl. A61K 9/51; A61K 45/00

(52) U.S. Cl. CPC A61K 9/5115; A61K 9/5123; A61K 9/5153; A61K 45/00

(57) ABSTRACT

According to an example aspect of the present invention, there are provided biomolecules encapsulated with Metal Organic Frameworks (MOFs) for use in intracellular delivery and controlled release of the biomolecules within cells, in vitro and in vivo. The invention also discloses the use of MOFs in combination with biomolecules for gene editing, cancer therapy and vaccine development.

### FIELD

The present invention relates to methods for encapsulation of biomolecules in Metal Organic Frameworks (MOFs), to MOF coated biomolecules for use in intracellular delivery and controlled release of the biomolecules in cells, in vitro and in vivo, and to a method for intracellular transfection and controlled release of biomolecules. The invention also discloses the use of MOFs in combination with biomolecules for gene editing, cancer therapy and vaccine development.

### BACKGROUND

Biomacromolecule species (such as nucleic acids, enzymes, proteins and even living cells) play important roles in many biological applications. Improving the robustness of these biomacromolecules is critical, but challenging in biotechnology, and if addressed, will significantly expand their applications in biocatalysis, biostorage, biomedical delivery, and biological vaccines etc. It is well known that living organisms can produce inorganic exoskeletons with excellent mechanical properties that can withstand harsh conditions. Inspired by these natural biomineralization phenomena, biomimetic mineralization is to adopt and transform the self-assembly processes to develop a general method of encapsulating bioactive molecules within protective exteriors (Nudelman & Sommerdijk, 2012) (10.1002/anie.201106715).

As biomimetic mineralization can solve the constraints of loading biological macromolecules into the MOFs pores during the preparation of MOFs-biomacromolecule composites and the adverse effects of solvents on enzyme structure, it has great potential for constructing immobilized biomacromolecules with higher loading and biological activity to further facilitate the fabrication and application of novel biocomposites. For example, compared to the free enzyme and urease encapsulated via the controlled co-precipitation method, biomimetic mineralization technique could extend the bioactive temperature range of enzyme and enhance the stability of enzyme (Liang et al, 2016) (10.1039/c5cc07577g). Further, it could avoid the utilization of polyvinylpyrrolidone as a capping agent, and thus it exhibits characteristics of low cost, easy preparation, high efficiency, and few wastes. Increasing efforts have been devoted for in situ growing MOFs exoskeleton around the biomacromolecules since the first report in 2014 by Lyu et al 10.1021/nl5026419). For biomimetic mineralization, MOFs are constructed from organic and inorganic components, thermally and chemically stable, can be directly grown on different biomacromolecules based on the strong interactions between the bio-interface and metal nodes under mild biocompatible conditions. Meanwhile, MOFs possess open architectures and large pore volumes, which can facilitate the selective transport of small molecules through the protective porous coating, enabling the selective interaction of the biomacromolecule with the external environment.

For example, patent application publications WO 2016/00032 A1, WO 2018/000043 A1 and WO 2019/227091 A1 relate to encapsulation of biomolecules in metal organic frameworks. Further attempts to prepare biomolecules coated with a MOF layer are disclosed i.a. by Balachandran et al (2021), Cheng et al (2020) and Yantao et al (2019).

Nowadays, MOFs-biomacromolecule composites are designed to retain activity with maximal loading and minimal leaching, and are typically produced either by surface adsorption, covalent grafting, cage inclusion or in situ synthesis. For the “in situ MOF synthesis” approach, nucleation, and growth of MOF formation and biomacromolecules immobilization take place simultaneously in a single step.

In the present invention, biomimetic mineralization technique allows in situ encapsulation of biomacromolecules within MOFs regardless the surface charges and sizes of biomolecules, and the tightly surrounding MOFs layer enables to significantly stabilize the biomacromolecules by conformational (Chen et al, 2019) (10.1002/anie.201813060). In terms of biological applications, the constructed MOFs-biomolecules can provide a range of applications in biocatalysts, sensing, protection, and storage by mineralizing various MOFs on different biomolecules. Among them, the applications of MOFs-biomacromolecules with the ability to release biomacromolecules in therapeutic delivery and genetic engineering would be highly desirable. The present invention provides the use of MOFs in combination with biomolecules i.a. for gene editing, cancer therapy, and vaccine development by providing methods for intracellular delivery and release of MOF coated biomolecules, in particular MOF coated biomacromolecules.

### SUMMARY OF THE INVENTION

The invention is defined by the features of the independent claims. Some specific embodiments are defined in the dependent claims.

The present invention is based on the concept of using simple biomineralization techniques to modulate metal-organic frameworks to encapsulate biomolecules (for example plasmids, anti-PD-L1 and mRNA), exploring the relationship between nanoparticle structure and molecular properties, such as CRISPR/Cas9 plasmids regulating the synthesis and encapsulation of ZIF-8, the corresponding gene editing through intracellular delivery for plasmid transfection and expression. Further, we have developed some specific synthetic methods and stimulation interventions to regulate the effective release of loadings through stimuli-responsive. For example, we have introduced photothermal conversion nanoparticles in the form of microfluidic-assisted biomineralization process to achieve effective release of Cas9 proteins and improve gene editing efficiency under the stimulation of near-infrared light. Still further, we have conducted intracellular enzymatic catalysis for MOF degradation by adding additional supplementary molecules to solve any slow intracellular release of loaded molecules. Meanwhile, appropriate metal ions and organic ligands have been selected for biomineralization according to the practical application to encapsulate various biological macromolecules, while the MOF decomposition components can assist in enhancing the biological activity of the loaded molecules, enabling customized biological application services.

Thus in its first aspect the present invention provides biomolecules encapsulated with stimuli-responsive non-cytotoxic Metal Organic Frameworks (MOFs) for use in intracellular delivery and controlled release of the biomolecules within cells.

According to a second aspect of the present invention, there is provided a method of preparing a biomolecule encapsulated with a stimuli-responsive coating layer of non-cytotoxic Metal Organic Framework (MOF), wherein the method comprises providing Metal Organic Framework precursor compounds, which comprise non-cytotoxic metal ions and organic ligands; and combining in an aqueous solution the biomolecule, the MOF precursor compounds and a stimulus sensitive agent to provide a stimuli-responsive layer of MOF on the biomolecule; or combining the biomolecule, the MOF precursor compounds and a stimulus sensitive agent in a microfluidic system to provide a layer of MOF on the biomolecule.

The invention also provides a method for intracellular transfection and controlled release of biomolecules within cells, wherein the method comprises the steps of: (i) providing biomolecules encapsulated with a stimuli-responsive non-cytotoxic Metal Organic Framework (MOF), and (ii) incubating the MOF encapsulated biomolecules with the cells, whereby the cells are transfected with the MOF encapsulated biomolecules. The intracellular release of the biomolecules may be triggered by external stimuli selected from light, heat, magnetism and any combinations thereof and/or by endogenous stimuli, such as intracellular pH, redox substances, enzymes or ATP.

A still further aspect of the present invention relates to the use of non-cytotoxic Metal Organic Framework (MOF) precursor compounds in combination with stimuli-sensitive material, in particular thermosensitive polymers or thermosensitive particles, in encapsulating biomolecules, wherein the MOF precursor compounds form a layer of MOF around the biomolecule, and the stimuli-sensitive material is included in the structure, preferably as an outer layer on the coated biomolecule, or the stimuli-sensitive material is used as a template of MOFs.

Embodiments of the invention comprise MOF encapsulated biomolecules, which comprise means for controlled or improved release of the biomolecule, wherein said means preferably comprise a stimulus sensitive polymer or other substance in the structure of the MOF-encapsulated biomolecule or stimulus sensitive particles as a template of MOFs. Thus the MOF encapsulated biomolecule may comprise for example positively charged polymers assembled in between the MOF framework and/or cell penetrating peptides to improve intracellular release of the biomolecules or loaded biomolecules.

Considerable advantages are obtained by the invention. First, the present invention can effectively trigger the formation of MOF through various biomolecules (including nucleic acids, enzymes and proteins) and the structure of the produced porous materials can be controlled by a biomimetic mineralisation process in the physiological environment. The present invention demonstrates that there is a critical synergy relationship between MOF precursors and biomolecules in the reaction solution.

Second, the biomimetic mineralisation of MOFs surrounding biomolecules forms porous nanoshells on their surfaces, providing unprecedented protection against biological, thermal, and chemical degradation to maintain their biological activity. The tuneable surface pore size promotes selective delivery of small molecules through the protective porous coating, allowing selective interaction of biomolecules with the external environment.

Third, the protective MOF coatings are responsive to internal and/or external stimuli such as intracellular pH changes or external signals such as light, heat and magnetism, enabling the controlled release of biomolecules from shells as bioactive drugs. Further, the intracellular release of biomolecules may be improved by suitable release improving structures in the MOF encapsulate biomolecule.

Further, this biomimetic approach, capable of protecting and delivering biologically active molecules, makes stability no longer a problem limiting the application of biomolecules, greatly broadening their application in biomedicine, biocatalysis, biovaccines, etc. In particular, the present invention provides methods and means for intracellular delivery and controlled release of biomacromolecules with a mass of 1000 Da or above, althought the method of the invention is applicable to all biomolecules.

Further features and advantages of the present technology will appear from the following description of some embodiments.

### BRIEF DESCRIPTION OF THE DRAWINGS

FIG. 1 shows the transmission electron microscopy (TEM) images of the morphologies of the composites formed with two different encapsulation strategies, P1-ZIF-8 (P1Z, FIG. 1a) and ZIF-8-P1 (ZP1, FIG. 1b) with 0 to 6 μl of P1 plasmids;

FIG. 2 shows the stability of P1 against Eco32I (EcoRV) degradation in

15 two structures by gel electrophoresis. FIG. 2 (a) The agarose gel electrophoresis image of EcoRV digestion assay: ladder, naked P1, EcoRV-treated P1, the releasing P1 from ZP1 and P1Z after EcoRV treatment. (b) DNA remains functionally intact during encapsulation and release-Fluorescence microscopy images of U2OS cells transfected with P1, with P1 previously encapsulated in ZP1 and P1Z, the scale bar=1000 μm. (c) Cumulative release profile of P1 from P1Z and ZP1 nanocarrier at different pH value, (dash line pH=7.4 and solid pH=5.5).

FIG. 3 shows endo/lysosomal escape of nanocarriers. The U2OS cell lines were incubated with Cy5.5-labeled PIZ and then cells were visualized with CLSM after 1, 2, 4 and 6 hours of incubation. CLSM images (63×) of U2OS cells after incubation with P1Z (red) for 1, 2, 4 and 6 h. Lysotracker@Green (green) was used to stain the acidic organelles (endosomes) and DAPI was used to stain the nucleus. Co-localization was indicated by yellow fluorescence, the scale bar=50 μm.

FIG. 4 shows the gene editing efficiency of the proposed nanovectors. Confocal immunofluorescent analysis of P1P2Z-treated U2OS cells using primary antibody (anti-paxillin antibody [Y113]) and secondary antibody (TRITC conjugated Goat Anti-Rabbit IgG (H+L)) showing colocalization with GFP signals, the scale bar=5 μm.

FIG. 5 shows the TEM of ZIF-8 (a), PD-L1@ZIF-8 (b) and PD-L1@ZIF-8-R837@PNcM (c).

FIG. 6 shows the thermal stability of αPD-L1 within ZIF-8 nanoshells. Temperature stability of PD-L1 alone (a) and released from PD-L1@ZIF-8 nanoparticles (b). (c) Cumulative release profile of PD-L1 from nanocarrier at different pH value. (d) Cumulative release profile of R837 from nanocarrier at different temperature.

FIG. 7 shows the antitumor efficacy of nanoparticles from tumor sizes in different groups. FIG. 7 (a) Tumor growth after systemic application of different treatment groups; (b) antitumor effect in terms of tumor growth.

FIG. 8 shows a three-dimensional microfluidic co-flow focusing device where the core-shell structure (PB@EuMOFs) is formed in a sandwich of two solutions due to the growth of EuMOFs on a PB hard template. FIG. 8 (a) Overview of 3D co-flow microfluidic device to prepare PB@EuMOFs core-shell nanoparticles and structures of every reactants; (b) Schematics of microfluidic and bulk synthesis methods of PB@RNP-EuMOFs composites. Fluidic 1 was the mixture of PB and Eu³⁺ and fluidic 2 was the mixture of GMP and RNP.

FIG. 9 (a) Water fluid patterns in microfluidic channel visualized by light microscope under different FRR. The RD zone was labeled with fluorescence dye and placed at the bottom of the corresponding FRR pictures; (b) TEM images of PB@EuMOFs prepared with different FRR, the concentration ratio of PB:Eu:GMP:Cas9 was 1:2:2.2; (c) TEM images of PB@EuMOFs prepared with different concentrations of reactants under the FRR=3. The white dotted line was used to highlight the EuMOFs not growing on PB and the red dotted line was used to highlight the thickness of EuMOFs layers.

FIG. 10 (a) Real-time temperature profiles of PB@EuMOFs at various concentration upon 808 nm laser irradiation (2 W/cm², 10 min); (b) Photothermal images of PB@EuMOFs solution at various concentration recorded after 10 min laser exposing; (c) Cumulative release profile of RNP from PB@RNP-EuMOFs under different temperature within 50 h; (d) NIR-triggered cumulative release profile of RNP from PB@RNP-EuMOFs at different temperature and different time points in grey boxes. Bars represent mean±SD (n=3).

FIG. 11. GFP gene editing efficiency of PB@EuMOFs and PB@RNP-EuMOFs in Hela/GFP cells under the condition with and without laser irritation detected by flow cytometry (a), GFP fluorescence intensity (b) and fluorescence microscopy (c). (d) PB@EuMOFs-mediated photo control of GFP editing efficiency detected by flow cytometry. The transfection, irradiation and gene-editing processes were illustrated and the laser irradiation time should be controlled temperature does not exceed 42° C.

### EMBODIMENTS

### Definitions

“Metal Organic Frameworks” (MOFs) are a class of compounds comprising metal ions or metal clusters coordinated by organic ligands to form one-, two-or three-dimensional structures. In the present context, the term “MOF” relates particularly to metal organic frameworks, which comprise non-toxic metals and non-toxic organic ligands. In particular, the MOFs within this disclosure comprise non-cytotoxic MOFs synthesized in an aqueous solution at room temperature and ambient pressure.

Typically the “framework” of MOFs is porous, comprising cavities in the form of cages connected by channels. The MOFs may be amorphous or crystalline, typically crystalline. Previously it has been found that it is possible to produce MOFs having a framework that encapsulates a biomolecule, where the biomolecule promotes the formation of the encapsulating framework.

In the present invention we present a biomimetic strategy that allows in situ tailored MOF biocomposites to control the nanostructures. The formed MOF coatings can be changed from homogeneous to heterogeneous structures by adding the plasmids in different precursors of MOFs. The effects of both embedding strategies of nanostructures on plasmid biofunctionality were studied and it was showed that the heterostructured nanoparticles could enhance the retention and protect the hosted plasmid. Based on the above synthetic strategy, we explored the intracellular delivery capability of the CRISPR/Cas9 Plasmid-ZIF system for Paxillin (PXN) knock-in via the HDR pathway. The study showed that these proof-of-concept functionalized MOF nanovectors can overcome different physical barriers to efficiently deliver CRISPR/Cas9 plasmids to cells, thereby effectively recovering the edited genes via HDR

Thus in its first embodiment the invention relates to biomolecules encapsulated with Metal Organic Framework (MOF) for use in intracellular delivery and controlled release of the biomolecules within cells.

The invention relates particularly to biomacromolecules with a mass of 1000 Da or above, althought the method of the invention is applicable to all biomolecules. Biomacromolecules typically include but are not limited to nucleic acids, peptides, proteins, including mRNA, plasmids, enzymes, antibodies, and Cas9/sgRNA ribonucleoprotein complexes (RNPs). Further examples of preferred biomolecules for the purposes of the invention are biomolecules selected from CRISPR/Cas9 plasmids; CRISPR/Cas13, CRISPR/Cas12; anti-PD-L1; mRNA; and Cas9/sgRNA ribonucleoprotein complexes (RNPs).

The biomolecules may provide a therapeutic or prophylactic effect on their own and/or be loaded with therapeutic or prophylactic agents. Typically the MOF encapsulated biomolecules for use according to the invention are used in various types of disease that need the intracellular delivery of biomolecules, including cancer therapy, gene therapy and gene editing, vaccine development, vaccine therapy, genetic diseases, tissue remodelling and/or tissue engineering, and combinations thereof.

Suitable metal ions that form part of a MOF structure are selected from non-toxic metal ions, in particular from non-cytotoxic MOF precursors, such as Ca²⁺, Mg²⁺, Zn²⁺, Fe²⁺, Fe³⁺, Cu²⁺, Eu³⁺, and Zr⁴⁺. Suitable organic ligands are selected from non-cytotoxic organic ligands, such as terephthalates, imidazoles, benzoates, carboxylates and combinations thereof.

In some embodiments, the MOFs are selected from zinc imidazolate frameworks (ZIFs), preferably from ZIF-8 and ZIF-90, more preferably ZIF-8, other Zn based MOFs, such as IRMOF-3, lanthanide-based MOFs, preferably EuBTC (Eu benzenetricarboxylate frameworks), Fe and/or Al based MOFs, such as MIL-53 and MIL-88B, Cu based MOFs, such as HKUST-1, Zr based MOFs such as UiO-66, UiO-66-NH₂ and UiO-67.

In one embodiment, Zn²⁺ ions and 2-methylimidazole (2-MIM) are selected as the raw materials for mineralisation, forming zeolitic imidazole framework-8 (ZIF-8) by coordination. The formed protective coating has a high surface area, exceptional chemical and thermal stability and negligible cytotoxicity, while being cost-effective and widely available.

However, also other types of MOFs (for example ZIF-90, Eu-BTC and HKUST-1) that can protect biomolecules have similar effects, demonstrating the generality and versatility of the biomimetic mineralisation approach.

The present invention is thus applicable to a variety of different MOFs, including amorphous MOFs and crystalline MOFs. In one embodiment of the invention, the MOFs are crystalline.

The MOF layer encapsulating the biomolecule has a tunable thickness based on needs. To facilitate the endocytosis said parameter is usually modulated to between 50 and 500 nm.

The encapsulation of biomolecules in MOF structures protects the biomolecules from enzymatic degradation and helps to maintain stability and bioactivity of the encapsulated biomolecules. Thus the present invention enables to tailor MOF-encapsulated bioarchitecture into controllable nanostructures via biomimetic mineralization, and emphasizes the significance of MOF structures for upholding the biological functionality of the obtained nanostructures. It was found that for example plasmids can be well distributed throughout the plasmid-MOF embedding structure and benefit from efficient protection against enzymatic degradation. The CRISPR/Cas9 plasmid-MOF system showed good loading, proper protection against enzymatic degradation and pH-responsive release of the plasmid, exhibiting better cellular endocytosis and efficient endo/lysosomal escape properties, making it outstanding for gene knock-in. Consequently, the present invention not only provides a fast, facile and low-cost method to load gene motifs in controlled nanostructures for efficient intracellular transfection, but also sheds light on the potential of MOF-based non-viral vectors in a variety of gene therapies.

The method according to the invention for preparing biomolecules with a stimuli-responsive coating layer of non-cytotoxic Metal Organic Framework (MOF) comprises the steps of (i) providing Metal Organic Framework precursor compounds, which comprise non-cytotoxic metal ions and organic ligands; and (ii) combining in an aqueous solution the biomolecule, the MOF precursor compounds and a stimulus sensitive agent to provide a layer of MOF on the biomolecule; or (iii) combining the biomolecule, the MOF precursor compounds and a stimulus sensitive agent in a microfluidic system to provide a layer of MOF on the biomolecule.

In an embodiment, where the biomolecules and the MOF precursor compounds are combined without a microfluidic system, the order of mixing the components may be of importance for optimizing the formation of MOF coated biomolecules. In one embodiment, the biomolecules and the MOF precursor compounds are combined in the aqueous solution by mixing first the biomolecules with the metal ions, followed by addition of the organic ligands. In another embodiment, the biomolecules and

MOF precursors are combined in the aqueous solution by mixing the biomolecules first with the organic ligands, followed by addition of the metal ions.

The molar amounts of biomolecules, metal ions and organic ligands have no fixed ratio and the actual ratio depends on the type of biomolecule and the capacity of the biomolecule to facilitate the formation of the MOF coating. In some embodiments, the molar ratio of metal ions to organic ligands is 1:1. In some embodiments, the molar ratio of metal ions, biomolecules and organic ligands can range from 1:1:100 to 100:1:1.

In an embodiment of the invention a substance, preferably a polymer or lipid, that has an opposite charge to the biomolecules is added during the preparation process to enhance the encapsulation and/or to complete the release of biomolecules inside the cells. Said substance may comprise for example PLGA-PEG/G0-C14, in particular for encapsulation of mRNA, polyethyleneimine (PEI), in particular for encapsulation of nucleic acids like plasmids, and polyvinylpyrrolidone (PVP), typically for encapsulation of enzymes and proteins. Preferably, said substance, for example a polymer or lipid, is added into the solution comprising the biomolecules, before mixing with metal ions or organic ligands.

### Stimulus Sensitive Release

The release of the biomolecules may be triggered by external stimuli selected from light, heat, magnetism and any combinations thereof, and/or by endogenous stimuli, such as intracellular pH, redox substances, enzymes or ATP. For said purpose the MOF encapsulated or the MOF coated biomolecules comprises means for stimulus-sensitive release of the biomolecule, wherein said means preferably comprise a stimulus sensitive polymer or other stimulus sensitive substance in the structure of the MOF-coated biomolecule or stimulus sensitive particles as a template of MOFs.

Endogenous stimuli, which are characteristic in the pathological areas of disease, include for example intracellular pH, redox substances, enzymes or ATP.

In an embodiment, where the MOF coated biomolecules comprise means for external stimulus-sensitive release of the biomolecules, said means preferably are sensitive to light, heat or both.

In a further preferred embodiment, the MOF coated biomolecules comprise means for thermal responsive release of the biomolecule, wherein said means preferably comprise a thermosensitive polymer in the structure of the MOF coated biomolecule, preferably as an outer layer on the MOF-coated biomolecule, or thermosensitive particle material as a template of MOFs.

Typically, the thermosensitive polymer is selected from poly(N-isopropyl acrylamide) (PNIPAAm), copolymers of PNIPAAm with poly(N,Ndiethylacrylamide) (PDEAAm), poly(N-vinylcaprolactone) (PVCL), PLGA, poly[2-(dimethylamino) ethyl methacrylate] (PDMAEMA), PEG, gelatin, chitosan, polysorbate and combinations thereof.

In one embodiment, the means for stimulus-sensitive release of the biomolecule may comprise stimulus sensitive particles as a template of MOFs, particularly thermosensitive particle material as a template of MOFs. For example, surface-activated nanoparticles can be hard templates and encapsulated in the MOFs.

The thermosensitive particle material preferably comprises thermosensitive nanoparticle material. Thermosensitive nanoparticle materials suitable for use in the present invention include but are not limited to selected from Au nanoparticles, PB (Prussian Blue) nanoparticles, transition metal semiconductor nanocrystals, like CuS nanoparticles, and magnetic nanoparticles, such as Fe₃O₄, and any combinations thereof.

Alternatively, the stimulus sensitive material may be for example a photosensitive polymer, preferably a photosensitive polymer selected from reversible addition-fragmentation chain transfer (RAFT) polymers and derivatives thereof.

In a still further embodiment, the MOF coated biomolecule may comprise positively charged polymers assembled in between the MOF framework and/or cell penetrating peptides to improve intracellular release of the biomolecules or loaded biomolecules, wherein said positively charged polymers are preferably selected from polyamine polymers, such as polyethyleneimine, and the cell penetrating peptides are preferably selected from TAT, Penetratin, Polyarginine, P22N, DPV3, DPV6 or combinations thereof.

A core-shell nanosystem based on the above disclosed biomineralization strategy was designed for thermo-induced tumor cascade immunotherapy after radiofrequency ablation. In the experimental part it is demonstrated that PD-L1@ZIF-8-R837@PNcM can be well dispersed in tumors after RFA and that the ZIF-8 shell protects αPD-L1 activity from hyperthermia. In addition, PD-L1@ZIF-8-R837@PNcM can release the immune adjuvant R837 through thermally triggered structural deformation of PNcM. Simultaneously, αPD-L1 is released from the pH-degraded ZIF-8 shell in tumor tissue, which further promotes the recognition and killing of tumor cells by T cells in synergistic with R837. The present experiments show that this triggered nanosystem, such as the post-RFA-triggered nanosystem, elicits potent tumor-specific immunity with significant suppression of metastatic tumors. Overall, the design of this integrated NPs provides a paradigm for the use of nanomaterials to promote tumor immunotherapy.

### Microfluidic System

In a further embodiment we have improved the invention by developing a microfluidic-assistant biomineralization strategy for MOFs and using it for efficient delivery and release of biomolecules, in particular for efficient delivery and remote regulation of CRISPR/Cas9 RNP gene editing.

Thus in one embodiment of the invention the biomolecule and the MOF precursor compounds are combined in a capillary microfluidic droplet system (chip), wherein the method comprises the steps of providing i) a first inner phase aqueous solution comprising the metal ions and a stimulus sensitive agent, preferably in combination with other agents thatthat enhance the intracellular delivery and release of the biomolecule, ii) a second inner phase aqueous solution comprising the biomolecule with the organic ligands, and iii) an outer phase comprising nonpolar oil; and generating microdroplets in a glass chip device, wherein a mixed aqueous solution of the two inner phases is squeezed into the immiscible outer phase of the oil by a microfluidic pump, the mixed aqueous solutions forming micrometer size droplets comprising the contents of the two aqueous solutions whereby a MOF layer is coated on top of the biomolecules inside the microdroplets.

In the microfluidic system the microdroplets are thus generated by a glass

chip device that may be fabricated via a simple assemble method. The droplet size can be regulated by adjusting the flow rate of the inner and outer phases. The structure of the chip allows for a direct connection between the droplet generation device and the plastic tubing, which prevents leakage and merging of the droplets. The encapsulation conditions in the microfluidic system are ambient temperature (room temperature) and pressure.

The first and second aqueous solutions in the microfluidic system are flow focused to form micrometer size droplets, containing all contents added in the two aqueous solutions. A MOF layer is coated on top of the biomolecules inside the microdroplets.

Typically, the first inner phase aqueous solution comprises metal ions selected from Ca²⁺, Mg²⁺, Zn²⁺, Fe²⁺, Fe³⁺, Cu²⁺, Eu³⁺, and Zr⁴⁺, while the second inner phase aqueous solution comprises non-toxic organic ligands selected from terephthalates, imidazoles, benzoates and carboxylates.

Outer phase typically comprises nonpolar oils such as mineral oil and HFE-7500.

In one embodiment of the invention the first inner phase aqueous solution comprises agents enhancing the encapsulation of the biomolecules, agents improving the release of the biomolecules inside the cells, or both.

Consequently, the first inner phase aqueous solution may comprise for example thermosensitive nanoparticle material, preferably selected from AuNP, Prussian Blue (PB), transition metal semiconductor nanocrystals, like CuS nanoparticles, magnetic nanoparticles, such as Fe₃O₄, and any combinations thereof, preferably PB nanoparticles.

The microfluidic system is particularly useful for preparing MOFs, which comprise organic ligands with poor water solubility. Such organic ligands are for example 2-Imidazolecarboxaldehyde and 2-Aminoterephthalic acid.

Therefore, the microfluidic system is particulary useful in the preparation of such MOF nanostructures as HKUST-1, UiO-66 (Zr), MIL-88B, ZIF-8, ZIF-90 and IRMOF-3.

The droplet-based reaction significantly reduces the reaction volume to nanoliter, to create a microenvironment with homogeneous heat transfer and fast molecular movement, therefore improving the efficiency of biomineralization. This method is especially useful when the type of MOF selected needs heat or solvent to form a layer on top of the biomolecular under normal condition as described above. The in-drop synthesis of MOF may enable the formation without solvent and without heat.

This strategy was verified by biomimetic growing thermal-responsive EuMOFs onto photothermal template PB and encapsulating RNP during MOFs crystallization in microfluidic channels. This strategy was more time efficient than the bulk encapsulation process and allowed higher reproducible encapsulation process, higher encapsulation efficiency and better protection for RNP in the presence of trypsin and SDS by simple adjusting of microfluidic parameters (flow rate or reactant concentration). Due to the photo-thermal conversion ability of PB and thermal-responsive degradation of the correspondings MOFs, such as EuMOFs, the nanocarrier offers an appealing avenue to control biomolecule release, such as RNP release, under NIR irritation. As a proof-of-concept, this strategy here combined effective delivery and precise control of CRISPR/Cas9 RNP-based gene editing, showing its great potential for biomedical therapy application.

As an example, a microfluidic-assistant MOFs biomineralization strategy was constructed and utilized for efficient CRISPR/Cas9 RNP delivery and NIR-responsive gene-editing remote control. By simply adjusting the microfluidic parameters (flow rate and reactant concentration), thermal-responsive degraded EuMOFs could grow on photothermal conversion template PB, regularly and encapsulated RNP during EuMOFs crystallization. Due to the combination of microfluidic technology and MOFs-based biomineralization, RNP encapsulated nanocarriers (PB@RNP-EuMOFs) possessed more uniform particle distribution, higher encapsulation efficiency and better RNP protect capacity than tradition bulk nanoprecipitation method which had more crystal defects in MOFs structure. Under NIR laser irradiation, the heat induced by PB conversion could induce the degradation of EuMOFs, resulting in promoted endosomal escape and effective RNP release. In addition, our strategy had successfully down-regulated the expression of targeted GFP gene via NIR light-activated gene-editing in vitro. The gene-editing activity could be programmed by exposure times turning, which shown higher editing efficiency (42%, three times and 47%, four times). Taken together, the present invention provides a proof-of-concept of microfluidic technology in MOFs biomineralization and its application in precise CRISPR/Cas9 gene-editing. The present invention thus offers one useful tool for CRISPR/Cas9 gene-editing-based precise biomedical therapy.

Therefore, in one preferred embodiment of the invention, the biomolecules are encapsulated with a stimuli-responsive MOF layer by combining the biomolecules, the MOF precursor compounds and a stimulus-sensitive agent in a microfluidic system. The invention may thus provide a stimuli-responsive polymer/nanoparticle embedded in a MOF matrix together with a material to facilitate delivery, with the aim of enhancing endocytosis of the final formulations as well as actively modulating the stimuli-responsive degradation of the MOF for controlled release of encapsulated biomolecules.

As disclosed above, the invention provides a method for intracellular transfection and controlled release of biomolecules within cells, wherein the method comprises the steps of: (i) providing biomolecules coated with a layer of non-toxic Metal Organic Framework (MOF), and (ii) incubating the MOF coated biomolecules with the cells, whereby the cells are transfected with the MOF coated biomolecules. The intracellular release of the biomolecules may be triggered by external stimuli selected from light, heat, magnetism and any combinations thereof and/or by endogenous stimuli, such as intracellular pH, redox substances, enzymes or ATP.

In one embodiment of the above method, the MOF coated biomolecules comprise a coating of a thermosensitive polymer, which is destabilized under application of heat, such as after radiofrequency ablation (RF), or the MOF coated biomolecules comprise thermal responsive template, such as PB.

Finally, the invention also provides the use of Metal Organic Framework (MOF) precursor compounds in combination with stimuli-sensitive material, in particular thermosensitive polymers or thermosensitive particles, in coating biomolecules, wherein the MOF precursor compounds form a layer of MOF around the biomolecule, and the stimuli-sensitive material is preferably included as an outer layer on the coated biomolecule or used as a template of MOFs.

As disclosed above, the stimuli-sensitive material may comprise for example a thermosensitive polymer in the structure of the MOF coated biomolecule, preferably as an outer layer on the MOF-coated biomolecule, or thermosensitive particle material, typically thermosensitive nanoparticles, as a template of MOFs.

It is to be understood that the embodiments of the invention disclosed are not limited to the particular structures, process steps, or materials disclosed herein, but are extended to equivalents thereof as would be recognized by those ordinarily skilled in the relevant arts. It should also be understood that terminology employed herein is used for the purpose of describing particular embodiments only and is not intended to be limiting.

Reference throughout this specification to one embodiment or an embodiment means that a particular feature, structure, or characteristic described in connection with the embodiment is included in at least one embodiment of the present invention. Thus, appearances of the phrases “in one embodiment” or “in an embodiment” in various places throughout this specification are not necessarily all referring to the same embodiment. Where reference is made to a numerical value using a term such as, for example, about or substantially, the exact numerical value is also disclosed.

As used herein, a plurality of items, structural elements, compositional elements, and/or materials may be presented in a common list for convenience. However, these lists should be construed as though each member of the list is individually identified as a separate and unique member. Thus, no individual member of such list should be construed as a de facto equivalent of any other member of the same list solely based on their presentation in a common group without indications to the contrary. In addition, various embodiments and example of the present invention may be referred to herein along with alternatives for the various components thereof. It is understood that such embodiments, examples, and alternatives are not to be construed as de facto equivalents of one another, but are to be considered as separate and autonomous representations of the present invention.

Furthermore, the described features, structures, or characteristics may be combined in any suitable manner in one or more embodiments. In the following description, numerous specific details are provided, such as examples of lengths, widths, shapes, etc., to provide a thorough understanding of embodiments of the invention. One skilled in the relevant art will recognize, however, that the invention can be practiced without one or more of the specific details, or with other methods, components, materials, etc. In other instances, well-known structures, materials, or operations are not shown or described in detail to avoid obscuring aspects of the invention.

### EXPERIMENTAL

### Example 1: Improving the in Vivo Transfection Efficiency of MOF-Embedded Plasmid-Like Biomolecules Through Controlled Embedding Structure

In the typical synthesis process, 0.5 μg to 3 μg lyophilized of Plasmid-1 (P1) was dispersed into 2 μL zinc nitrate solution (67.5 mg·mL⁻¹) then stirred for 5 min before added into 8 μL 2-methyl imidazole solution (2-MIM, 463.75 mg·mL⁻¹). The mixture was aged for 1 h, and the formed P1-ZIF-8 (P1Z) biocomplexes was collected by centrifugation at 8000 rpm, and then washed, sonicated, and centrifuged three times to remove loosely adsorbed plasmids.

The similar synthesis process, where P1 was dispersed into 8 μL 2-MIM solution then stirred for 5 min, followed by adding 2 μL zinc acetate solution. The mixture was aged for 1 h, and the formed ZIF-8-P1 (ZP1) biocomplexes was collected by centrifugation at 8000 rpm, and then washed, sonicated, and centrifuged three times to remove loosely adsorbed plasmids.

P1P2-ZIF-8 (P1P2Z) nanostructures were synthesized with the similar process. The P1 and P2 mixed with 1:1 ratio and 2 μL zinc nitrate solution were well mixed and stirred for 5 min at room temperature before added into 8 μL 2-MIM solution slowly. 1 h later, the final product was obtained after centrifugation at 8000 rpm for 5 min, washed and dispersed into water.

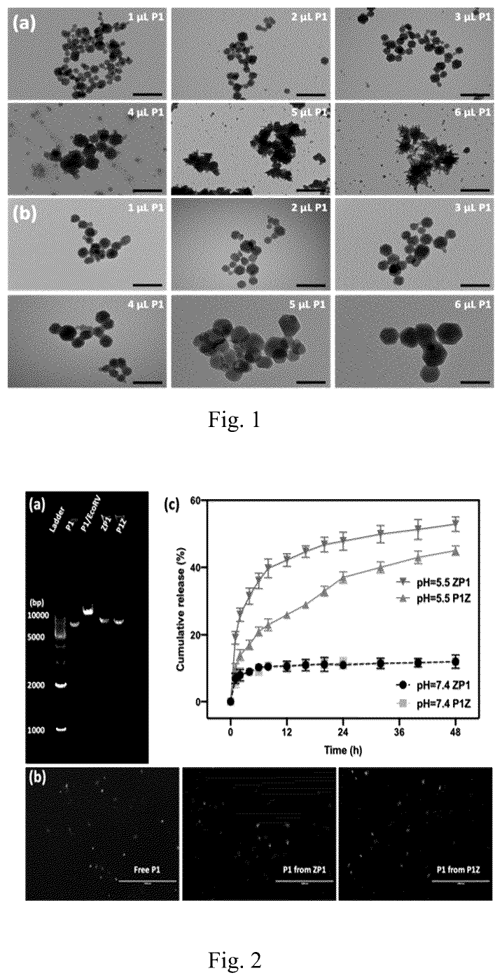

The evolution of the embedding structure of the composites (P1Z and PZ1) was studied by transmission electron microscope (TEM in FIG. 1).

As shown in FIG. 1a, when the biomineralization was modulated with 1 to 3 μl of plasmid, the complex formed a homogeneous rhombohedral dodecahedron with a diameter of about 100 nm. By adding more plasmids to 4 and 5 μl, the nanostructures gradually evolved into irregular polyhedron with spiky surfaces. The increase in plasmid dose (to 6 μl) resulted in morphological evolution to decussation of approximately 200 nm size. However, in the ZP1 system (FIG. 1b), the nucleation rates of a series of nanoparticles were much slower due to the coordination competition effect between the plasmid and the 2-MIM ligand, in which the morphologies of the nanoparticles remained uniformly rhombohedron dodecahedron, but the size of the ZP1 nanostructures increased from 100 nm to greater than 200 nm.

For the digestion reaction shown in FIG. 2a, the nuclease-free water (16 μL) and 10× buffer R (2 μL) were mixed together and then adding 1 μL pure P1, released P1 from P1Z and ZP1, following by adding EcoRV (2 μL). The solution for P1 without EcoRV was taken as control group. Every solution was mixed gently and spun down for a few seconds. Afterwards, they were incubated overnight at 37° C. water bath. After that, the samples (10 μL) mixed with 6× loading buffer (2 μL) were loaded into comb wells carefully. The Fast Ruler High Range DNA ladder was loaded into the first lane of the gel. The gel was placed into the gel box and run it at 130 V for 100 min. Finally, the gel was photographed with a ChemiDoc imaging system (Bio-Rad) to visualize the DNA fragments.

Lipid-based standard cellular transfected with P1, P1 released from P1Z, and ZP1 (FIG. 2b) to check any damage in the encapsulation process that would have negative effects on the functional activity.

pH-responsive release of P1 from P1Z and ZP1 nanostructures in FIG. 2c: The in vitro release of encapsulated plasmid from nanocarriers and the effect of pH on the release profiles were determined by suspending the P1Z and ZP1 in 20 μL PBS buffer with different pH (7.4 and 5.5), respectively. All suspensions were placed in a shaker (GrantGLS400). The amount of released plasmid was determined by removing the supernatant after centrifugation (8000 rpm, 5 min) and replacing it with a new buffer at the collecting time points. Then the release profile was measured using NanoDrop 2000c Fluorospectrometer (ThermoFisher, USA) and all the measurements were performed in triplicate.

As can be seen in FIG. 2a, the free P1 severely cleaved into a linearized DNA conformation and left lower band in P1/EcoRV. P1 was extracted from both ZP1 and P1Z nanostructures after EcoRV treatment, where the DNA backbone was not digested and found to be in the same form as free P1. Lipid-based standard cellular transfected with P1, P1 released from P1Z, and ZP1, all showed fluorescence (FIG. 2b). That means the plasmid does not suffer any kind of damage in the encapsulation process that would have negative effects on the functional activity. Furthermore, pH-responsive release behaviour of P1 in both structures was investigated by immersing them in PBS solutions with different pH values and their cumulative release from nanocarriers was detected by UV-visible spectrophotometry in FIG. 2c.

Cellular endo-/lysosomal escape assay. The U2OS cells were seeded in confocal dishes (2*10⁵ cells/dish) and incubated overnight for attachment. The plasmids-loaded nanocarrier (Cy5.5 labelled P1Z) was dispersed into DMEM and added to each dish. LysoTracker@Green probe was used to stain endosomes according to the manufacture protocol. At the determined time point (1, 2, 4 and 6 h), the cells were fixed with 4% PFA and further stained with DAPI. Then the endosomal escape ability of the nanocarrier were measured with CLSM.

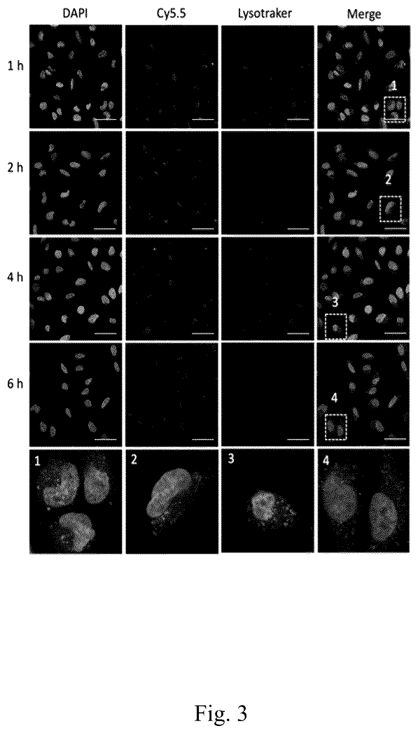

As can be seen in FIG. 3, after 1 h of incubation, a clear overlap (yellow) between the red fluorescence of the nanocarriers and green fluorescence of the endo/lysosomes can be observed, indicating that P1Z can be localized in the endosomes after internalization into U2OS cells. After 6 h of incubation, in the absence of gene expression, the intracellular Cy5.5-labelled P1Z showed an increase in red fluorescence intensity and a greater overlap with the nucleus-specific blue fluorescence (FIG. 3, enlarged 4), while the green fluorescence became weaker as the escape of nanostructures led to a reduction in intracellular lysosomes.

Immunofluorescence test. After the PFA fixation, the cells were treated with glycine (1 M in PBS, 30 min) to quench background autofluorescence coming from the PFA and then permeabilized with 0.1% Triton X-100 in PBS for 5 min at room temperature. Permeabilization removes the cellular membrane lipids which helps the antibodies accessibility to the interior of the cell for better targeting of the paxillin protein. Cells were stained with primary antibodies at 1:100 dilution of the Rabbit recombinant monoclonal Paxillin antibody in PBS and kept at +4° C. overnight. The cells were then washed twice with PBS and incubated with secondary antibodies, Tetramethylrhodamine (TRITC) conjugated Goat Anti-Rabbit IgG (H+L) (Novex) at dilution of 1:500 (45 min, RT). After the incubation, cells were washed three times with PBS and later observed by using a confocal microscope (Zeiss, LSM 880). Helium-Neon 543 nm laser line was used for TRITC, Argon 488 nm laser line for paxillin-GFP and 405 nm diode laser line for DAPI.

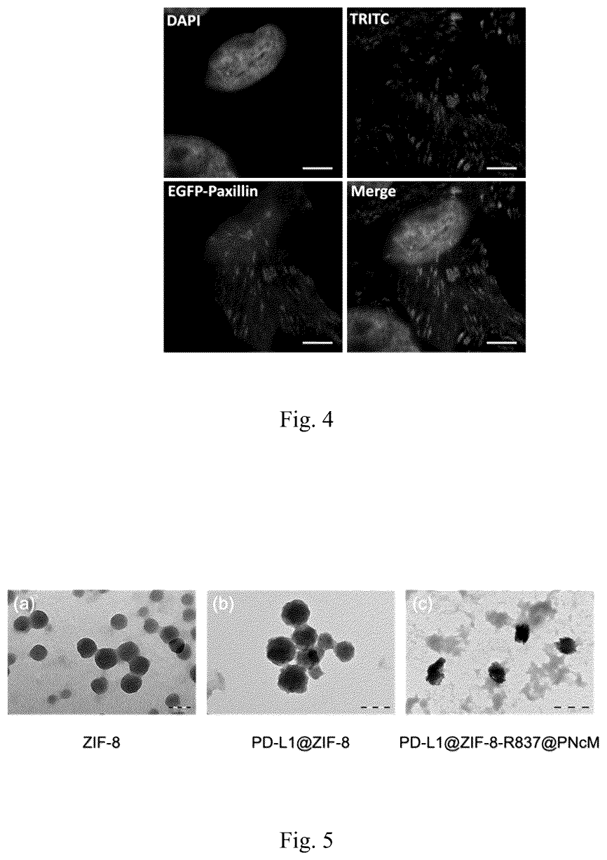

The gene editing efficiency of the proposed nanovectors is shown in FIG. 4. A clear colocalization of distinct fluorescent channels (paxillin, red and GFP, green) was obtained when the edited cells were stained with monoclonal paxillin antibody and TRITC-conjugated goat anti-rabbit lgG (expected to label both GFP-tagged and untagged paxillin protein derivatives in the same cell).

### Example 2: A Study of Cascade Anti-Tumour Immune Response After Radiofrequency Ablation Based on Bionanomineralised Nanoparticles

ZIF-8 material preparation: the method described by Liang et al., 2015 can be employed: prepare 4 mL each of Zn²⁺ solution (40 mM) and 2-MIM solution (160 mM), add equal amounts of Zn²⁺ solution to 2-MIM solution drop by drop, stir and mix, then leave to mineralize for 60 min, after the crystals have completed growth, centrifuge at 10,000 rpm for 5 min, discard the supernatant, and resuspend the precipitate well with 500 μL of ultrapure water.

PD-L1@ZIF-8 nanoparticle preparation: Prepare 2.5 mg/ml of PD-L1 antibody, add 2 mL to 2-MIM solution, stir gently for 5 min at 4° C. to mix thoroughly, add the same volume of Zn²⁺ solution drop by drop to the 2-MIM solution containing PD-L1 antibody, and leave it for 60 min for mineralization. After the crystals have grown, centrifuge at 10,000 rpm for 5 min, discard the supernatant and reserve the precipitate.

PD-L1@ZIF-8-R837@PNcM nanoparticle preparation: Weigh 5 mg of PNcM powder, dissolve it in 1 mL of ultrapure water, add 2 mg of R837 drug powder, add the solution to the above centrifuged sediment, and stir well to disperse it, then keep it overnight at 4° C. until PD-L1@ZIF-8 has fully loaded the drug and coated polymer.

As shown in FIG. 5, TEM picture showed the successful preparation of the ZIF-8 NPs with an average size of approximate 100 nm. From TEM image of αPD-L1@ZIF-8 (FIG. 5b), the encapsulation of αPD-L1 had no effect on the formation of the nanostructures. FIG. 5c revealed the successful preparation of the polymer coated core-shell PD-L1@ZIF-8-R837@PNcM nanoparticles.

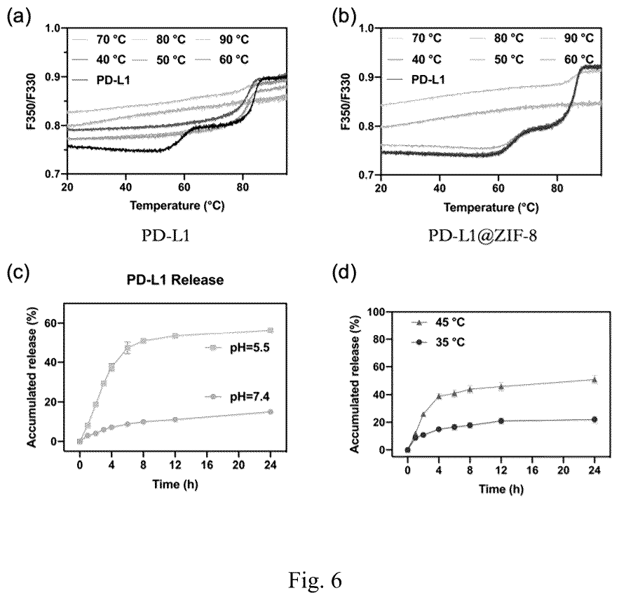

To investigate the thermoprotective effect of the ZIF-8 shell on αPD-L1, αPD-L1 and αPD-L1@ZIF-8 were placed at different temperatures for 10 min then measured by nano differential scanning fluorimetry (FIGS. 6a and 6b).

The nano differential scanning fluorimetry showed (FIGS. 6a and 6b) that in the αPD-L1 group, most of the αPD-L1 was denatured. As we expected, αPD-L1 remained in the αPD-L1@ZIF-8 group up to 80 degrees due to the protective effect of the ZIF-8 encapsulation, which maintained their 3D structure. Furthermore, encapsulation and removal of the ZIF-8 shell had a negligible effect on the activity of αPD-L1.

pH triggered the release of αPD-L1 antibody: The dialysis bag (14 kDa) containing 1 mL of αPD-L1@ZIF-8 (1 mg·mL⁻¹) solution was immersed in different pH of PBS solution. The antibody release experiment was conducted at the constant temperature water bath in FIG. 6c (37° C., 100 rpm). At the indicated time points, the dialyzed solution was replaced by the fresh one. Finally, the antibody concentration in dialysate was determined with the BCA Protein Assay Kit.

ZIF-8 could be degraded by PBS due to the lower pH, the αPD-L1 antibody release profile showed in FIG. 6c, the release rate of αPD-L1 was slow in physiological environment (pH=7.4). Comparatively, the release rate of αPD-L1 at pH=5.5 was quite fast.

We next examined the temperature-responsive drug release of PD-L1@ZIF-8-R837@PNcM nanoparticles. R837-loaded were PD-L1@ZIF-8-R837@PNcM immersed in 2 mL (pH=7.4 and 5.5) phosphate buffered saline (PBS) at 30 and 45° C. with gentle shaking, respectively. At predetermined time intervals, PBS was taken out and replaced with an equal volume of fresh PBS.

FIG. 6d shows the in vitro release profiles of R837 loaded with PD-L1@ZIF-8-R837@PNcM in PBS buffer at temperatures of 30 and 45° C. At low temperatures, drug release reached an equilibrium within 8 hours, except for an initial small burst effect due to the surface attachment of R837, and then remained nearly static for several hours due to the blocking effect of the loose polymer weaving. In contrast, the rapid release of R837 at 45° C. was consistent with the contraction of the PNcM brushes at high temperatures, and the system exhibited rapid release behaviour.

Cytoxicity. Next, the cytotoxicity effects of nanoparticles and drugs on HCC cells were assessed by the methylthiazolium tetrazolium (MTT) assay.

Blood biochemistry and blood routine assays. BALB/c mice were randomly divided into two groups (n=3) and injected intravenously with PBS and PD-L1@ZIF-8-R837@PNcM, respectively. 3 days later, blood samples were collected by cardiac puncture and analysed using a blood biochemistry analyser and an automated haematology analyser.

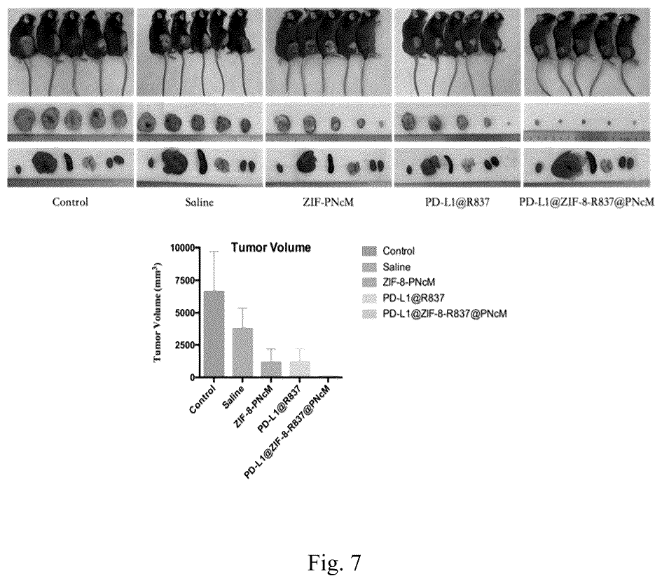

Evaluation of Antitumor Effect: Tumor-bearing mice were randomly divided into five groups: control, PBS, ZIF-PNcM, PD-L1@R837, PD-L1@ZIF-8-R837@PNcM in FIG. 7. Mice were treated 8 and 15 days after radiofrequency ablation with nanoparicles and drugs though intravenous injection. The tumor sizes and body weight were recorded every other day. The tumor volume (V) was calculated according to the formula: V=L×W²/2, where L and W respectively represent the longest and shortest diameter of the tumor. On day 22, some mice were sacrificed, and tumor and major organs were sectioned for H&E. In addition, the intratumoral cytokines (CCl1, CCL2, IL-1β, IFN-γ, and IL-6) were analyzed by ELISA.

FIG. 7 shows the antitumor efficacy of nanoparticles from tumor sizes in different groups. Compared to the control and PBS groups, tumors in the carrier (ZIF-PNcM) group were only slightly inhibited, indicating that the vector was not significantly toxic because of its low tumor accumulation. αPD-L1+R837 group showed a slight anti-tumor effect, indicating that the anti-tumor effect of the single treatment modality was limited. PD-L1@ZIF-8-R837@PNcM showed the best anti-tumor effect, which was attributed to the controlled release of the drug and enhanced immunotherapy.

Consequently, in the above we have developed a biomineralized nanoparticle for anti-hepatocellular carcinoma (HCC) immune response after incomplete RF ablation (iRFA), which has multiple functions in immune stimulation, including in situ stimuli-responsive release of the immune adjuvant (imiquimod (R837)) and pH-controlled release of anti-PD-L1 (αPD-L1) antibodies. The PD-L1@ZIF-8-R837@PNcM are loaded with αPD-L1 via pH-sensitive ZIF-8 as a carrier and further coated with a thermosensitive polymer for prolonged cycle time and temperature responsive release of R837. ZIF-8 protects αPD-L1 from the physiological environment and heat by immobilizing it in the structures. Once injected at the tumour site, NPs destabilise the polymer membrane under residual heat after iRFA and release R837. Subsequently, the pH within the tumour degrades the ZIF-8 shell and large amounts of αPD-L1 are released from the NPs to act directly on the target tumour cells, promoting T cell recognition and killing of tumour cells with the aid of adjuvants.

### Example 3: Microfluidic-Assisted Biomineralization of Biomolecules in a Stimulus-Responsive Metal-Organic Framework

Preparation of PB: In a typical process, PVP (3.0 g) and K₃[Fe(CN)₆] (226.7 mg) were dissolved into water (40 mL) to form a clear aqueous solution by magnetic stirring. Then 35 μL concentrated hydrochloric acid were added. The reaction solution was kept stirring for 30 min and then heated at 80° C. for 20 h. After aging, the precipitates were collected by centrifugation and washed three times with Milli-Q water and ethanol. The PB products were dispersed in water for further use.

Fabrication of Microfluidic chip: The 3D microfluidic co-flow focusing chip was fabricated by assembling two borosilicate glass capillaries (World Precision Instruments Ltd, UK) on a glass slide. The two glass capillaries with outer diameter of around 1000 and 1100 μm, respectively, were named as inner and outer capillaries. In brief, one end of the inner capillary was tapered using a magnetic glass microelectrode horizontal needle puller (P-31, Narishige Co., Ltd, Japan) and polished using sandpaper (Indasa Rhynowet, Portugal) until the cross-section became flattened. Then the inner tapered capillary was inserted into the outer capillary and coaxially aligned. After fixing them on the glass slide, one hypodermic needle was be situated to the outer capillary and sealed with transparent epoxy resin.

### Preparation of PB-EuMOFs:

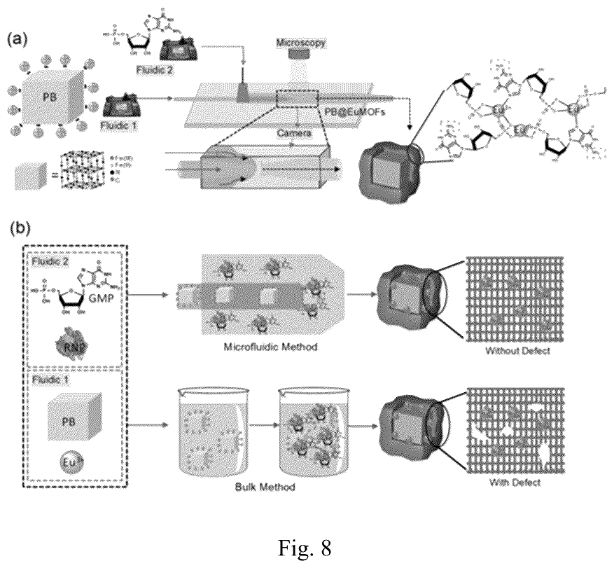

In microfluidic system: The PB-EuMOFs core-shell nanoparticles were prepared by the microfluidic 3D co-flow device at room temperature in FIG. 8. In general, PB and Eu³⁺ mixed together and kept stirring for 5 min to form one stable system by electrostatic interaction, which served as the inner phase. The GMP aqueous solution was selected as the outer fluid. The inner and outer fluids were separately pumped into the microfluidic device, in which the inner fluid were focused by the outer continuous fluid. The flow rate of the different liquids was controlled by pumps (PHD 2000, Harvard Apparatus, USA). In this procedure, EuMOFs particle formed and coated outside of PB immediately. The resulting product was collected by centrifugation and washed with distilled water three times to remove any residues. In order to optimize the physicochemical properties of the prepared core-shell nanoparticles, several process variables and formulation parameters were evaluated, such as the total flow rate of inner and outer fluids, the flow ratio between the inner and outer fluids, and the concentration of PB, Eu³⁺, and GMP.

In bulk method: By comparation, we have prepared the PB-EuMOFs nanoparticles using a bulk method. In brief, the PB and Eu³⁺ mixed solution was added dropwise into the GMP solution with the same concentration in microfluidic system.

FIG. 8 shows the three-dimensional microfluidic co-flow focusing device where the core-shell structure (PB@EuMOFs) is formed in a sandwich of two solutions due to the growth of EuMOFs on a PB hard template. For CRISPR/Cas9 ribonucleoprotein (RNP) encapsulation, the same microfluidic co-flow focusing device was utilized in FIG. 8b.

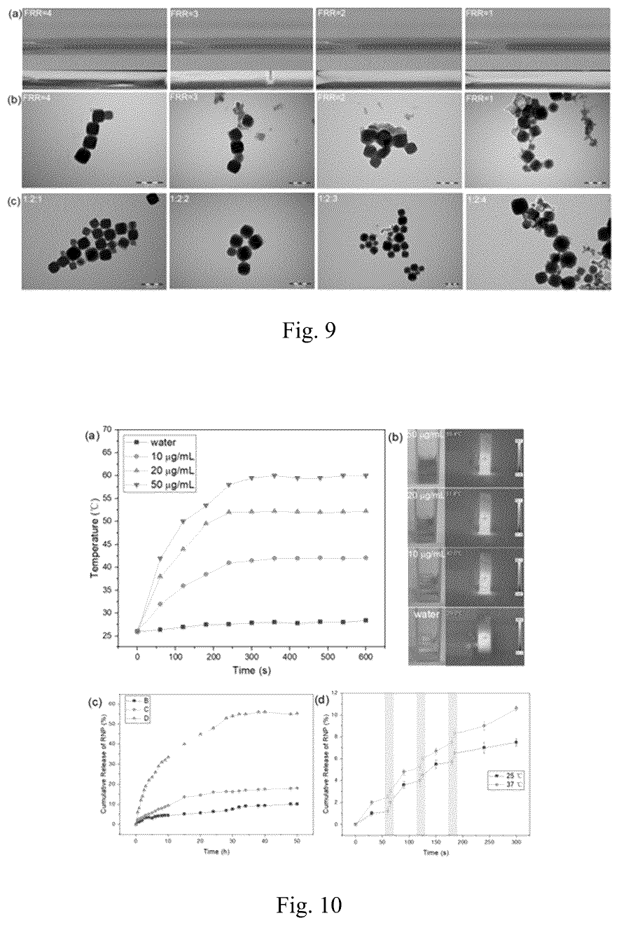

We defined the two input channels and corresponding flow rates as fluidic 1 (Q1) and fluidic 2 (Q2). The flow rate ratio (FRR) was defined as the ratio of flow between Q2 and Q1. TEM determined the prepared PB@EuMOFs by using different FRR in FIG. 9.

FIG. 9 shows the width of the reaction-diffusion (RD) zone formed

between two-reagent streams (where the diffusive mixing occurs). We used pure water with different dyes to visualize this process (FIG. 9a). Thus, the microfluidic procedure allows tailoring the core-shell structure of the prepared PB@EuMOFs through the variation of RD conditions. FIG. 9b displays the TEM images of the prepared PB@EuMOFs using different flow rate ratio (FRR). In all cases, EuMOFs were immediately formed at the interface between Eu³⁺ and GMP, whereas the core-shell structure shown significant difference.

The NIR-controlled release of the obtained RNP@PB-EuMOFs was investigated upon radiation by the 808 nm laser in FIG. 10. In brief, the collected RNP@PB-EuMOFs were dispersed into PBS and irradiated with NIR light for different time periods. The amount of released RNP was determined by removing the supernatant after centrifugation and replacing it with a new buffer at the collecting time points. Then the releasing profiles was measured using UV-Vis spectrophotometer according to the absorption wavelength of Cy5.5. As control, the releasing behavior without NIR irradiation was also carried out under the same procedures.

As shown in FIG. 10a, the temperature of PB@EuMOFs solution quickly increased and achieved a plateau of 42° C. under the concentration of 10 μg/mL for 6 min. The final temperature under different concentration was recorded with an infrared thermal camera (FIG. 10b). Then cumulative releasing profiles were determined and performed under different temperatures, which shown thermal responsive RNP release property (FIG. 10c). As shown in FIG. 10d, the RNP release profile with laser-stimulus displayed burst release phenomenon in both different temperature solution.

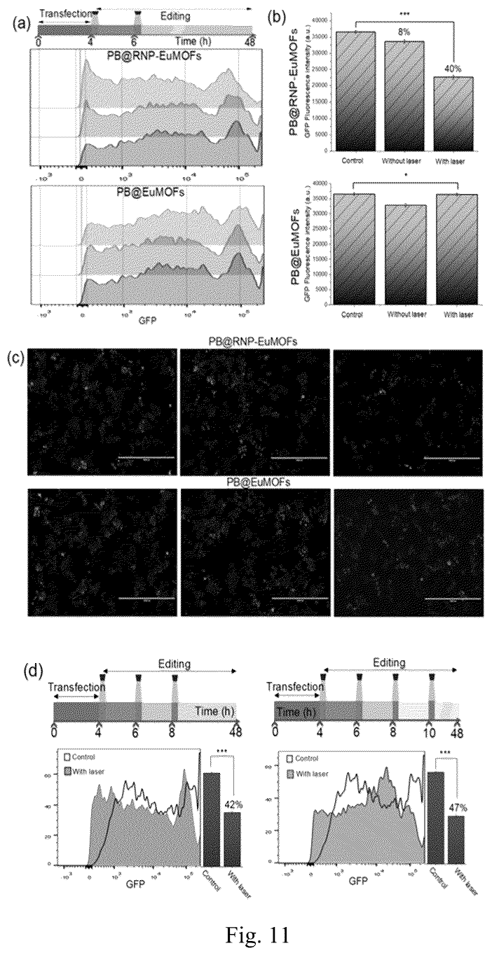

In vitro GFP disruption assay: Hela/GFP cells were seeded into 96-well plate (4×10³ cells/well) the day before particle adding in FIG. 11. PB@RNP-EuMOFs were prepared fresh and added to the wells, leading to the final amount of Cas9 was 250 ng and sgRNA was 50 ng. After incubation, 808 nm laser irradiation (2 W·cm⁻¹, 10 s) was performed at determined time points (two times: 4 h and 6 h; three times: 4 h, 6 h and 8 h; four times: 4 h, 6 h, 8 h and 10 h). Infrared thermometer was used to monitor the temperature of cell culture medium during laser exposing and control the temperature up to 42° C. The cells were kept at 37° C. and incubated for 48 h. Afterwards, the cells were analyzed fluorescence microscopy. The GFP gene disruption efficiency was quantified by flow cytometry.

FIG. 11 displays the gene editing ability of the nanocarriers through targeted DNA cleavage and NHEJ-induced repair. As shown in FIGS. 11a and 11b, the intensity of GFP fluorescence in Hela/GFP cells decreased by 40% with the laser irradiation in the group of PB@RNP-EuMOFs. Fluorescence microscopy images also confirmed the quenching of GFP signals after PB@RNP-EuMOFs incubating and laser exposing (FIG. 11c). As reflected by FIG. 11d, the intensity of GFP fluorescence decreased more (efficiency: 42%) following three times irradiation comparing with the result in FIG. 11a.

While the forgoing examples are illustrative of the principles of the present invention in one or more particular applications, it will be apparent to those of ordinary skill in the art that numerous modifications in form, usage and details of implementation can be made without the exercise of inventive faculty, and without departing from the principles and concepts of the invention. Accordingly, it is not intended that the invention be limited, except as by the claims set forth below.

The verbs “to comprise” and “to include” are used in this document as open limitations that neither exclude nor require the existence of also un-recited features. The features recited in depending claims are mutually freely combinable unless otherwise explicitly stated. Furthermore, it is to be understood that the use of “a” or “an”, that is, a singular form, throughout this document does not exclude a plurality.

The invention is further described by the following embodiments:
1. Biomolecules coated with a layer of Metal Organic Framework (MOF) for use in intracellular delivery and controlled release of the biomolecules within cells.
2. The biomolecules for use according to embodiment 1 wherein said biomolecules are selected from biomacromolecules with a mass of 1000 Da or above, preferably from nucleic acids, peptides, proteins, including mRNA, plasmids, enzymes, antibodies, and Cas9/sgRNA ribonucleoprotein complexes (RNPs).
3. The biomolecules for use according to embodiment 1 or 2, wherein said biomolecules are selected from CRISPR/Cas9 plasmids; CRISPR/Cas13, CRISPR/Cas12; anti-PD-L1; mRNA; and Cas9/sgRNA ribonucleoprotein complexes (RNPs).
4. The biomolecules for use according to any one of the preceding embodiments, wherein the biomolecule provides a therapeutic or prophylactic effect and/or is loaded with therapeutic or prophylactic agents.
5. The biomolecules for use according to any one of the preceding embodiments, wherein the release of the biomolecules is triggered by external stimuli selected from light, heat, magnetism and any combinations thereof, and/or by endogenous stimuli, such as intracellular pH, redox substances, enzymes or ATP.
6. The biomolecules for use according to any one of the preceding embodiments, wherein the MOF coated biomolecules comprise means for stimulus-sensitive release of the biomolecule, wherein said means preferably comprise a stimulus sensitive polymer or other substance in the structure of the MOF-coated biomolecule or stimulus sensitive nanoparticles as a template of MOFs.
7. The biomolecules for use according to any one of the preceding embodiments, wherein the MOF coated biomolecules comprise means for thermal responsive release of the biomolecule, wherein said means preferably comprise a thermosensitive polymer in the structure of the MOF coated biomolecule, preferably as an outer layer on the MOF-coated biomolecule, or thermosensitive particle material as a template of MOFs.
8. The biomolecules for use according to embodiment 7, wherein the thermosensitive polymer is selected from poly(N-isopropyl acrylamide) (PNIPAAm), copolymers of PNIPAAm with poly(N,Ndiethylacrylamide) (PDEAAm), poly(N-vinylcaprolactone) (PVCL), PLGA, poly[2-(dimethylamino) ethyl methacrylate] (PDMAEMA), PEG, gelatin, chitosan, polysorbate and combinations thereof.
9. The biomolecules for use according to embodiment 7, wherein the thermosensitive particle material is selected from Au nanoparticles, PB (Prussian Blue) nanoparticles, transition metal semiconductor nanocrystals, like CuS nanoparticles, and magnetic nanoparticles, such as Fe₃O₄, and any combinations thereof.
10. The biomolecules for use according to any one of the preceding embodiments, wherein the MOF coated biomolecule comprises a photosensitive polymer, preferably a photosensitive polymer selected from reversible addition-fragmentation chain transfer (RAFT) polymers and derivatives thereof.
11. The biomolecules for use according to any one of the preceding embodiments, wherein the MOF coated biomolecule comprises positively charged polymers assembled in between the MOF framework and/or cell penetrating peptides to improve intracellular release of the biomolecules or loaded biomolecules, wherein said positively charged polymers are preferably selected from polyamine polymers, such as polyethyleneimine, and the cell penetrating peptides are preferably selected from TAT, Penetratin, Polyarginine, P22N, DPV3, DPV6 or combinations thereof.
12. The biomolecules for use according to any one of the preceding embodiments, wherein the Metal Organic Framework is selected from MOFs formed from non-toxic MOF precursors, such as non-toxic metal ions selected from Ca²⁺, Mg²⁺, Zn²⁺, Fe²⁺, Fe³⁺, Cu²⁺, Eu³⁺, and Zr⁴⁺, and non-toxic organic ligands selected from terephthalates, imidazoles, benzoates, carboxylates and combinations thereof.
13. The biomolecules for use according to any one of the preceding embodiments, wherein the Metal Organic framework is selected from zinc imidazolate frameworks (ZIFs), preferably from ZIF-8 and ZIF-90, more preferably ZIF-8, other Zn based MOFs, such as IRMOF-3, lanthanide-based MOFs, preferably EuBTC (Eu benzenetricarboxylate frameworks), Fe and/or Al based MOFs, such as MIL-53 and MIL-88B, Cu based MOFs, such as HKUST-1, Zr based MOFs such as UiO-66, UiO-66-NH₂ and UiO-67.
14. The biomolecules for use according to any one of the preceding embodiments, wherein the Metal Organic Framework is amorphous or crystalline, preferably crystalline.
15. The biomolecules for use according to any one of the preceding embodiments in various types of diseases that need the delivery of biomolecules, including cancer therapy, gene therapy and gene editing, vaccine development, vaccine therapy, genetic diseases, tissue remodelling and/or tissue engineering and combinations thereof.
16. A method of preparing a biomolecule with a coating layer of Metal Organic Framework (MOF), the method comprising:
   providing Metal Organic Framework precursor compounds, which comprise metal ions and organic ligands;
   combining in an aqueous solution the biomolecule and the MOF precursor compounds to provide a layer of MOF on the biomolecule; and optionally including a stimulus sensitive agent or some other agent that provides certain functionality, such as enhancing the intracellular delivery and release, in the structure of the MOF-coated biomolecule; or
   combining the biomolecule and the MOF precursor compounds in a microfluidic system to provide a layer of MOF on the biomolecule.
17. The method according to embodiment 16, wherein the metal ions are selected from non-toxic metal ions, preferably from Ca²⁺, Mg²⁺, Zn²⁺, Fe²⁺, Fe³⁺, Cu²⁺, Eu³⁺, and Zr⁴⁺, and the organic ligands are non-toxic organic ligands, preferably selected from terephthalates, imidazoles, benzoates, carboxylates and combinations thereof.
18. The method according to embodiment 16 or 17, wherein the biomolecules and the MOF precursor compounds are combined in the aqueous solution by
   mixing first the biomolecules with the metal ions, followed by addition of the organic ligands; or
   mixing the biomolecules first with the organic ligands, followed by addition of the metal ions.
19. The method according to any one of embodiments 16 to 18, wherein the molar ratio of metal ions, biomolecules and organic ligands ranges from 1:1:100 to 100:1:1.
20. The method according to any one of embodiments 16 to 19, wherein a substance, preferably a polymer or lipid, that has an opposite charge to the biomolecules is added during the preparation process to enhance the encapsulation and/or to complete the release of biomolecules inside the cells.
21. The method according to embodiment 20, wherein said substance comprises PLGA-PEG/G0-C14, in particular for mRNA, PEI, in particular for nucleic acids like plasmids, and polyvinylpyrrolidone (PVP), typically for enzymes and proteins.
22. The method according to embodiment 16, wherein the biomolecule and the MOF precursor compounds are combined in a microfluidic droplet system, the method comprising the steps of
   providing i) a first inner phase aqueous solution comprising the metal ions, optionally in combination with other agents that provide a certain function, ii) a second inner phase aqueous solution comprising the biomolecule with the organic ligands, and iii) an outer phase comprising nonpolar oil;
   generating microdroplets in a glass chip device, wherein the droplet size can be regulated by adjusting the flow rate of the inner and outer phases;
   the first and second aqueous solutions form micrometer size droplets comprising the contents of the two aqueous solutions, whereby a MOF layer is coated on top of the biomolecules inside the microdroplets.
23. The method according to embodiment 22, wherein the first inner phase aqueous solution comprises agents enhancing the encapsulation of the biomolecules, agents improving the release of the biomolecules inside the cells, or both.
24. The method according to embodiment 22 or 23, wherein the first inner phase aqueous solution comprises thermosensitive nanoparticle material, preferably selected from AuNP, Prussian Blue (PB), transition metal semiconductor nanocrystals, like CuS nanoparticles, magnetic nanoparticles, such as Fe₃O₄, and any combinations thereof, preferably PB nanoparticles.
25. The method according to any one of embodiments 22 to 24, wherein the first inner phase aqueous solution comprises metal ions selected from Ca²⁺, Mg²⁺, Zn²⁺, Fe²⁺, Fe³⁺, Cu²⁺, Eu³⁺, and Zr⁴⁺, and the second inner phase aqueous solution comprises non-toxic organic ligands selected from terephthalates, imidazoles, benzoates and carboxylates.
26. The method according to any one of embodiments 22 to 25, wherein the MOFs comprise homogeneous nanostructures of MOFs, including HKUST-1, UiO-66 (Zr), MIL-88B, ZIF-8, ZIF-90 and IRMOF-3.
27. A method for intracellular transfection and controlled release of biomolecules within cells, comprising the steps of:
   providing biomolecules coated with a layer of non-toxic Metal Organic Framework (MOF);
   contacting the MOF coated biomolecules with the cells, whereby the cells are transfected with the MOF coated biomolecules;
      wherein the intracellular release of the biomolecules is triggered by external stimuli selected from light, heat, magnetism and any combinations thereof, and/or by endogenous stimuli, such as intracellular pH, redox substances, enzymes or ATP.
28. The method according to embodiment 27, wherein the MOF coated biomolecules comprise a coating of a thermosensitive polymer, which is destabilized under application of heat, such as after radiofrequency ablation (RF), or the MOF coated biomolecules comprise thermal responsive template, such as PB.
29. The method according to embodiment 27 or 28, wherein the loaded biomolecule works as a prophylactic or therapeutic agent in cells.
30. Use of Metal Organic Framework (MOF) precursor compounds in combination with stimuli-sensitive material, in particular thermosensitive polymers or nanoparticles, in coating biomolecules, wherein the MOF precursor compounds form a layer of MOF around the biomolecule, and the stimuli-sensitive material is preferably included as an outer layer on the coated biomolecule or used as a template of MOFs.
31. The use according to embodiment 30, wherein the stimuli-sensitive material comprises a thermosensitive polymer in the structure of the MOF coated biomolecule, preferably as an outer layer on the MOF-coated biomolecule, or thermosensitive nanoparticle material as a template of MOFs.

### INDUSTRIAL APPLICABILITY

At least some embodiments of the present invention find industrial application in pharmaceutical and diagnostic industry, providing for example genome editing tools, anticancer drugs, vaccine development and vaccine therapy agents, pharmaceutical agents, as well as diagnostic agents.

### ACRONYMS LIST

2-MIM 2-methylimidazole
APTEOS (3-aminopropyl)triethoxysilane
Cas9 CRISPR-associated protein-9 nuclease
Cy5.5 Sulfo-Cyanine5.5 NHS ester
CLSM confocal laser scanning microscopy
CRISPR clustered regularly interspaced short palindromic repeats
CTAC cetyltrimethylammonium chloride
CuS copper sulfide nanoparticles
DLS dynamic light scattering
DMEM Dulbecco's modified eagle's medium
GFP green fluorescent protein
LCST low critical solution temperature
MFI mean fluorescent intensity
MOF metal organic framework
pCas9 GFP-Cas9-paxillin_gRNA
pPXN AICSDP-1: PXN-EGFP
PBS phosphate-buffered saline
PNcM Poly(N-isopropylacrylamide-co-methacrylic acid)
sgRNA single guide RNA
TEM transmission electron microscopy
TEOS tetraethoxysilane
ZIF zeolitic imidazolate framework

### CITATION LIST

### Patent Literature

WO 2016/00032 A1
WO 2018/000043 A1
WO 2019/227091

### Non Patent Literature

Balachandran, Y.L., Li, X. and Jiang, X, 2021. Integrated Microfluidic Synthesis of Aptamer Functionalized Biozeolitic Imidazolate Framework (BioZIF-8) Targeting Lymph Node and Tumor. Nano Lett. 2021, 21, 1335-1344.
Cheng, Q., Gao, F., Yu, W-Y., Zou, M-Z, Ding, X-L., Li, M-J., Cheng, S-X, and Zhang, X-Z, 2020. Near-Infrared Triggered Cascade of Antitumor Immune Responses Based on the Integrated Core-Shell Nanoparticle. Adv. Funct. Mater. 2020, 30, 2000335.
Nudelman, F. and Sommerdijk, N.A., 2012. Biomineralization as an inspiration for materials chemistry. Angewandte Chemie International Edition, 51(27), pp.6582-6596.
Liang, K., Coghlan, C.J., Bell, S.G., Doonan, C. and Falcaro, P., 2016. Enzyme encapsulation in zeolitic imidazolate frameworks: a comparison between controlled co-precipitation and biomimetic mineralisation. Chemical Communications, 52(3), pp.473-476.
Lyu, F., Zhang, Y., Zare, R.N., Ge, J. and Liu, Z., 2014. One-pot synthesis of protein-embedded metal-organic frameworks with enhanced biological activities Nano letters, 14(10), pp.5761-5765.
Chen, G., Huang, S., Kou, X., Wei, S., Huang, S., Jiang, S., Shen, J., Zhu, F. and Ouyang, G., 2019. A convenient and versatile amino-acid-boosted biomimetic strategy for the nondestructive encapsulation of biomacromolecules within metal-organic frameworks. Angewandte Chemie International Edition, 58(5), pp.1463-1467.
Liang, K., Ricco, R., Doherty, C.M., Styles, M.J., Bell, S., Kirby, N., Mudie, S., Haylock, 5 D., Hill, A.J., Doonan, C.J. and Falcaro, P., 2015. Biomimetic mineralization of metal-organic frameworks as protective coatings for biomacromolecules. Nature communications, 6(1), pp.1-8.
Yantao, L., Zhang, K., Liu, P., Chen, M., Zhong, Y., Ye, Q., Wei, M.Q., Zhao, H., and Tang, Z, 2019. Encapsulation of Plasmid DNA by Nanoscale Metal-Organic Frameworks for Efficient Gene Transportation and Expression. Adv. Mater. 2019, 31, 1901570.

1. Biomolecules encapsulated with stimuli-responsive non-cytotoxic Metal Organic Frameworks (MOFs) for intracellular delivery and controlled release of the biomolecules within cells.

2. The biomolecules according to claim 1 wherein said biomolecules are selected from biomacromolecules with a mass of 1000 Da or above, and wherein said biomolecules are selected from the group consisting of nucleic acids, peptides, proteins, including mRNA, plasmids, enzymes, antibodies, and Cas9/sgRNA ribonucleoprotein complexes (RNPs).

3. The biomolecules according to claim 1, wherein said biomolecules are selected from the group consisting of CRISPR/Cas9 plasmids; CRISPR/Cas13, CRISPR/Cas12; anti-PD-L1; mRNA; and Cas9/sgRNA ribonucleoprotein complexes (RNPs).

4. The biomolecules according to claim 1, wherein the biomolecules provide a therapeutic or prophylactic effect and/or are loaded with therapeutic or prophylactic agents.

5. The biomolecules according to claim 1, wherein the release of the biomolecules is triggered by external stimuli selected from light, heat, magnetism, or any combinations thereof, and/or by endogenous stimuli, and wherein the endogenous stimuli are intracellular pH, redox substances, enzymes or ATP.

6. The biomolecules according to claim 1, wherein the MOF encapsulated biomolecules comprise means for stimulus-sensitive release of the biomolecules, and wherein said means comprises a stimulus sensitive polymer in the structure of the MOF-coated biomolecules or stimulus sensitive nanoparticles as a template of MOFs.

7. The biomolecules according to claim 1, wherein the MOF encapsulated biomolecules comprise means for thermal responsive release of the biomolecule, wherein said means comprise a thermosensitive polymer as an outer layer on the MOF-encapsulatd biomolecules, or thermosensitive particle material as a template of MOFs.

8. The biomolecules according to claim 7, wherein the thermosensitive polymer is selected from the group consisting of poly(N-isopropyl acrylamide) (PNIPAAm), copolymers of PNIPAAm with poly(N,Ndiethylacrylamide) (PDEAAm), poly(N-vinylcaprolactone) (PVCL), PLGA, poly[2-(dimethylamino) ethyl methacrylate] (PDMAEMA), PEG, gelatin, chitosan, polysorbate, and combinations thereof.

9. The biomolecules according to claim 7, wherein the thermosensitive particle material is selected from the group consisting of Au nanoparticles, PB (Prussian Blue) nanoparticles, transition metal semiconductor nanocrystals, magnetic nanoparticles, and any combinations thereof.

10. The biomolecules according to claim 1, wherein the MOF encapsulated biomolecules comprise a photosensitive polymer, and wherein the photosensitive polymer is selected from the group consisting of reversible addition-fragmentation chain transfer (RAFT) polymers and derivatives thereof.

11. The biomolecules according to claim 1, wherein the MOF encapsulated biomolecules comprise positively charged polymers assembled in between the MOF framework and/or cell penetrating peptides to improve intracellular release of the biomolecules or loaded biomolecules, and wherein said positively charged polymers are selected from polyamine polymers, and the cell penetrating peptides are selected from TAT, Penetratin, Polyarginine, P22N, DPV3, DPV6 or combinations thereof.

12. The biomolecules according to claim 1, wherein the Metal Organic Frameworks are selected from MOFs formed from non-cytotoxic MOF precursors, wherein the non-cytotoxic MOF precursors comprise non-cytotoxic metal ions or non-cytotoxic organic ligands, wherein the non-cytotoxic metal ions are selected from the group consisting of Ca²⁺, Mg²⁺, Zn²⁺, Fe²⁺, Fe³⁺, Cu²⁺, Eu³⁺, and Zr⁴⁺, and wherein the non-cytotoxic organic ligands are selected from the group consisting of terephthalates, imidazoles, benzoates, carboxylates and combinations thereof.

13. The biomolecules according to claim 1, wherein the Metal Organic Frameworks are selected from the group consisting of zinc imidazolate frameworks (ZIFs), ZIF-8, ZIF-90, Zn based MOFs, IRMOF-3, lanthanide-based MOFs, EuBTC (Eu benzenetricarboxylate frameworks), Fe and/or Al based MOFs, MIL-53, MIL-88B, Cu based MOFs, HKUST-1, Zr based MOFs, UiO-66, UiO-66-NH₂ and UiO-67.

14. (canceled)

15. (canceled)

16. The biomolecules according to claim 1, wherein the biomolecules are biomolecules encapsulated by microfluidic-assisted synthesis of stimuli-responsive MOFs.

17. A method of preparing a biomolecule encapsulated with a stimuli-responsive coating layer of non-cytotoxic Metal Organic Framework (MOF), the method comprising:
providing Metal Organic Framework precursor compounds, which comprise metal ions and organic ligands;
combining in an aqueous solution the biomolecule and the MOF precursor compounds to provide a layer of a Metal Organic Framework (MOF) on the biomolecule; and further including a stimulus sensitive agent and optionally an agent that enhances the intracellular delivery and release of the MOF encapsulated biomolecule; or
combining the biomolecule, the MOF precursor compounds and the stimulus sensitive agent in a microfluidic system to provide a layer of MOF on the biomolecule.

18. The method according to claim 17, wherein the metal ions comprise non-toxic metal ions selected from the group consisting of from Ca²⁺, Mg²⁺, Zn²⁺, Fe²⁺, Fe³⁺, Cu²⁺, Eu³⁺, and Zr⁴⁺, and wherein the organic ligands comprise non-toxic organic ligands selected from the group consisting of terephthalates, imidazoles, benzoates, carboxylates and combinations thereof.

19. The method according to claim 17, wherein the biomolecule and the MOF precursor compounds are combined in the aqueous solution by:
mixing first the biomolecule with the metal ions, followed by addition of the organic ligands; or
mixing the biomolecule first with the organic ligands, followed by addition of the metal ions.

20. (canceled)

21. The method according to claim 17, wherein a substance that has an opposite charge to the biomolecule is added during the preparation process to enhance the encapsulation and/or to complete the release of the biomolecule inside the cells.

22. The method according to claim 21, wherein said substance comprises PLGA-PEG/G0-C14 for encapsulation of mRNA, polyethyleneimine (PEI) for encapsulation of nucleic acids like plasmids, and polyvinylpyrrolidone (PVP) for encapsulation of enzymes and proteins.

23. The method according to claim 17, wherein the biomolecule and the MOF precursor compounds are combined in a microfluidic droplet system, the method comprising the steps of:
providing: i) a first inner phase aqueous solution comprising the metal ions and a stimulus-sensitive agent, optionally in combination with other agents that enhance the intracellular delivery and release; ii) a second inner phase aqueous solution comprising the biomolecule with the organic ligands; and iii) an outer phase comprising a nonpolar oil;
generating microdroplets in a glass chip device, wherein the droplet size is regulated by adjusting the flow rate of the inner and outer phases; and
wherein the first and second aqueous solutions form micrometer size droplets comprising the contents of the two aqueous solutions, whereby a MOF layer is coated on top of the biomolecules inside the microdroplets.

24. The method according to claim 23, wherein the first inner phase aqueous solution n comprises agents enhancing the encapsulation of the biomolecule, agents improving the release of the biomolecules inside the cells, or both.

25. The method according to claim 23, wherein the first inner phase aqueous solution comprises a thermosensitive nanoparticle material selected from the group consisting of AuNP, Prussian Blue (PB) nanoparticles, transition metal semiconductor nanocrystals, magnetic nanoparticles, and any combinations thereof.

26. (canceled)

27. The method according to claim 23, wherein the MOFs comprise homogeneous nanostructures of MOFs selected from the group consisting of HKUST-1, UiO-66 (Zr), MIL-88B, ZIF-8, ZIF-90 and IRMOF-3.

28. A method for intracellular transfection and controlled release of biomolecules within cells, comprising the steps of:
providing biomolecules encapsulated with a stimuli-responsive non-cytotoxic Metal Organic Framework (MOF); and
contacting the MOF encapsulated biomolecules with cells, whereby the cells are transfected with the MOF encapsulated biomolecules;
wherein the intracellular release of the biomolecules is triggered by external stimuli selected from the group consisting of light, heat, magnetism and any combinations thereof, and/or by endogenous stimuli, intracellular pH, redox substances, enzymes or ATP.

29. The method according to claim 28, wherein the MOF coated biomolecules comprise a coating of a thermosensitive polymer, which is destabilized under application of heat, or the MOF encapsulated biomolecules comprise a thermal responsive template.

30. The method according to claim 28, wherein the biomolecules work as a prophylactic or therapeutic agent in cells or are loaded with therapeutic or prophylactic agents.

31. A combination comprising non-cytotoxic Metal Organic Framework (MOF) precursor compounds in combination with thermosensitive polymers or nanoparticles, in encapsulating biomolecules, wherein the MOF precursor compounds form a layer of a Metal Organic Framework (MOF) around the biomolecules, and wherein the thermosensitive polymers are included as an outer layer on the encapsulated biomolecules or the thermosensitive nanoparticles are used as a template of the Metal Organic Framework.

32. (canceled)

33. The method of claim 28, wherein the MOF-encapsulated biomolecules are used in the delivery of biomolecules for cancer therapy, gene therapy and gene editing, vaccine development, vaccine therapy, genetic diseases, tissue remodelling and/or tissue engineering and combinations thereof.

34. The biomolecules according to claim 1, wherein the Metal Organic Frameworks comprise ZIF-8.
